SEQUENCE LISTING (86 sequences)

SEQ ID NO: 1 - protein, 8 aa, Homo sapiens
VKKDSGFQ

SEQ ID NO: 2 - protein, 7 aa, Homo sapiens
MNQLRGK

SEQ ID NO: 3 - protein, 11 aa, Homo sapiens
SCHTGLGRSAG

SEQ ID NO: 4 - protein, 11 aa, Homo sapiens
YFGYSGAFKCL

SEQ ID NO: 5 - protein, 15 aa, Homo sapiens
QVPSHTVVARSMGGK

SEQ ID NO: 6 - protein, 15 aa, Homo sapiens
HFGKDKSKEFQLFSS

SEQ ID NO: 7 - protein, 15 aa, Homo sapiens
MYLGYEYVTAIRNLR

SEQ ID NO: 8 - protein, 15 aa, Homo sapiens
KKSASDLTWDNLKGK

SEQ ID NO: 9 - protein, 15 aa, Homo sapiens
NIPMGLLYNKINHCR

SEQ ID NO: 10 - protein, 15 aa, Homo sapiens
LCMGSGLNLCEPNNK

SEQ ID NO: 11 - protein, 15 aa, Homo sapiens
GYYGYTGAFRCLVEK

SEQ ID NO: 12 - protein, 7 aa, Homo sapiens
HAVVTRK

SEQ ID NO: 13 - protein, 11 aa, Homo sapiens
TDCSGNFCLFR

SEQ ID NO: 14 - protein, 15 aa, Homo sapiens
KYLGEEYVKAVGNLR

SEQ ID NO: 15 - protein, 15 aa, M. catarrhalis
MGYGMALSKINLHNR

SEQ ID NO: 16 - protein, 15 aa, M. catarrhalis
AMGYGMALSKINLHN

SEQ ID NO: 17 - protein, 14 aa, M. catarrhalis
MGYGMALSKINLHN

SEQ ID NO: 18 - protein, 15 aa, M. catarrhalis
GPVGGVFYNGTTTAK

SEQ ID NO: 19 - protein, 15 aa, M. catarrhalis
WNLGPVGGVFYNGTT

SEQ ID NO: 20 - protein, 12 aa, M. catarrhalis
GPVGGVFYNGTT

SEQ ID NO: 21 - protein, 15 aa, M. catarrhalis
FMTDVANRRNRFSEV

SEQ ID NO: 22 - protein, 15 aa, M. catarrhalis
AVKYKGHWDFMTDVA

SEQ ID NO: 23 - protein, 15 aa, M. catarrhalis
KGHWDFMTDVANRRN

SEQ ID NO: 24 - protein, 15 aa, M. catarrhalis
DFMTDVANRRNRFSE

SEQ ID NO: 25 - protein, 6 aa, M. catarrhalis
FMTDVA

SEQ ID NO: 26 - protein, 15 aa, M. catarrhalis
AGWYYGASSKDEYNR

SEQ ID NO: 27 - protein, 15 aa, M. catarrhalis
FSEVKENSQAGWYYG

SEQ ID NO: 28 - protein, 6 aa, M. catarrhalis
AGWYYG

SEQ ID NO: 29 - protein, 15 aa, M. catarrhalis
FSNLQDRHKGNVTKT

SEQ ID NO: 30 - protein, 4 aa, M. catarrhalis
FSNL

SEQ ID NO: 31 - protein, 15 aa, M. catarrhalis
NFKEKKLTGKLFSNL

SEQ ID NO: 32 - protein, 15 aa, M. catarrhalis
KKLTGKLFSNLQDRH

SEQ ID NO: 33 - protein, 15 aa, M. catarrhalis
GKLFSNLQDRHKGNV

SEQ ID NO: 34 - protein, 6 aa, M. catarrhalis
GKFSNL

SEQ ID NO: 35 - protein, 15 aa, M. catarrhalis
DANIHGNRFRGSATA

SEQ ID NO: 36 - protein, 8 aa, M. catarrhalis
DANIHGNR

SEQ ID NO: 37 - protein, 15 aa, M. catarrhalis
KTERYDIDANIHGNR

SEQ ID NO: 38 - protein, 15 aa, M. catarrhalis
YDIDANIHGNRFRGS

SEQ ID NO: 39 - protein, 11 aa, M. catarrhalis
YDIDANIHGNR

SEQ ID NO: 40 - protein, 15 aa, Artificial Sequence
  TbpB-binding peptides
KKSASDLTWDNLKGK

SEQ ID NO: 41 - protein, 15 aa, Artificial Sequence
  TbpB-binding peptides
NIPMGLLYNKINHCR

SEQ ID NO: 42 - protein, 15 aa, Artificial Sequence
  TbpB-binding peptides
LCMGSGLNLCEPNNK

SEQ ID NO: 43 - protein, 15 aa, Artificial Sequence
  TbpB-binding peptides
GYYGYTGAFRCLVEK

SEQ ID NO: 44 - protein, 15 aa, Artificial Sequence
  TbpB-binding peptides
CHLARAPNHAVVTRK

SEQ ID NO: 45 - protein, 15 aa, Artificial Sequence
  TbpB-binding peptides
GSNVTDCSGNFCLFR

SEQ ID NO: 46 - protein, 15 aa, Artificial Sequence
  TbpB-binding peptides
TDCSGNFCLFRSETK

SEQ ID NO: 47 - protein, 15 aa, Artificial Sequence
  TbpB-binding peptides
KYLGEEYVKAVGNLR

SEQ ID NO: 48 - protein, 15 aa, N. meningitidis
FYKHAASEKDFSNKK

SEQ ID NO: 49 - protein, 15 aa, N. meningitidis
PSRQLPASGKVIYKG

SEQ ID NO: 50 - protein, 15 aa, N. meningitidis
VIYKGVWHFVTDTKK

SEQ ID NO: 51 - protein, 15 aa, H. influenzae
AALNLFDRNKPSLLN

SEQ ID NO: 52 - protein, 15 aa, H. influenzae
APNSNENRHGQKYVY

SEQ ID NO: 53 - protein, 15 aa, H. influenzae
IQSWSLRDLPNKKFY

SEQ ID NO: 54 - protein, 15 aa, H. influenzae
SALPVGGVATYKGTW

SEQ ID NO: 55 - protein, 15 aa, H. influenzae
YKGTWSFITAAENGK

SEQ ID NO: 56 - protein, 15 aa, H. influenzae
RNSGGGQAYSRRSAT

SEQ ID NO: 57 - protein, 16 aa, H. influenzae
FTVNNFGTKKLTGGLY

SEQ ID NO: 58 - protein, 15 aa, H. influenzae
TDANKSQNRTHKLYD

SEQ ID NO: 59 - protein, 15 aa, H. influenzae
GKFLAHDKKVLGVFS

SEQ ID NO: 60 - protein, 15 aa, Artificial Sequence
  TbpB N-lobes of Bovine Pathgens
MVKWCAIGHQERTKC

SEQ ID NO: 61 - protein, 15 aa, Artificial Sequence
  TbpB N-lobes of Bovine Pathgens
CAIGHQERTKCDRWS

SEQ ID NO: 62 - protein, 15 aa, Artificial Sequence
  TbpB N-lobes of Bovine Pathgens
HQERTKCDRWSGFSG

SEQ ID NO: 63 - protein, 7 aa, Artificial Sequence
  bTF Binding Regions of the TbpB N-lobes of Bovine Pathgens
HQERTKC

SEQ ID NO: 64 - protein, 15 aa, Artificial Sequence
  TbpB N-lobes of Bovine Pathgens
KTSDANINWNNLKDK

SEQ ID NO: 65 - protein, 15 aa, Artificial Sequence
  TbpB N-lobes of Bovine Pathgens
ANINWNNLKDKKSCH

SEQ ID NO: 66 - protein, 11 aa, Artificial Sequence
  bTf Binding Regions of the TbpB N-lobes of Bovine Pathgens
ANINWNNLKDK

SEQ ID NO: 67 - protein, 15 aa, Artificial Sequence
  TbpB N-lobes of Bovine Pathgens
NSNERYYGYTGAFRC

SEQ ID NO: 68 - protein, 15 aa, Artificial Sequence
  TbpB N-lobes of Bovine Pathgens
RYYGYTGAFRCLVEK

SEQ ID NO: 69 - protein, 11 aa, Artificial Sequence
  bTf Binding Regions of the TbpB N-lobes of Bovine Pathgens
RYYGYTGAFRC

SEQ ID NO: 70 - protein, 15 aa, Artificial Sequence
  TbpB N-lobes of Bovine Pathgens
NTDGNNNEAWAKNLK

SEQ ID NO: 71 - protein, 15 aa, Artificial Sequence
  TbpB N-lobes of Bovine Pathgens
NNNEAWAKNLKKENF

SEQ ID NO: 72 - protein, 7 aa, Artificial Sequence
  bTf Binding Regions of the TbpB N-lobes of Bovine Pathgens
NLKKENF

SEQ ID NO: 73 - protein, 15 aa, Artificial Sequence
  TbpB N-lobes of Bovine Pathgens
AWAKNLKKENFEVLC

SEQ ID NO: 74 - protein, 11 aa, Artificial Sequence
  bTf Binding Regions of the TbpB N-lobes of Bovine Pathgens
NNNEAWAKNLK

SEQ ID NO: 75 - protein, 15 aa, Artificial Sequence
  TbpB N-lobes of Bovine Pathgens
NLKKENFEVLCKDGT

SEQ ID NO: 76 - protein, 3 aa, Artificial Sequence
  bTf Binding Regions of the TbpB N-lobes of Bovine Pathgens
NLK

SEQ ID NO: 77 - protein, 15 aa, Artificial Sequence
  TbpB N-lobes of Bovine Pathgens
CHLARGPNHAVVSRK

SEQ ID NO: 78 - protein, 15 aa, Artificial Sequence
  TbpB N-lobes of Bovine Pathgens
RGPNHAVVSRKDKAT

SEQ ID NO: 79 - protein, 15 aa, Artificial Sequence
  TbpB N-lobes of Bovine Pathgens
HAVVSRKDKATCVEK

SEQ ID NO: 80 - protein, 7 aa, Artificial Sequence
  bTf Binding Regions of the TbpB N-lobes of Bovine Pathgens
HAVVSRK

SEQ ID NO: 81 - protein, 15 aa, Artificial Sequence
  TbpB N-lobes of Bovine Pathgens
SRKDKATCVEKILNK

SEQ ID NO: 82 - protein, 3 aa, Artificial Sequence
  bTf Binding Regions of the TbpB N-lobes of Bovine Pathgens
SRK

SEQ ID NO: 83 - protein, 15 aa, Artificial Sequence
  TbpB N-lobes of Bovine Pathgens
RDDTKCLASIAKKTY

SEQ ID NO: 84 - protein, 15 aa, Artificial Sequence
  TbpB N-lobes of Bovine Pathgens
KCLASIAKKTYDSYL

SEQ ID NO: 85 - protein, 11 aa, Artificial Sequence
  bTf Binding Regions of the TbpB N-lobes of Bovine Pathgens
KCLASIAKKTY

SEQ ID NO: 86 - protein, 15 aa, Artificial Sequence
  TbpB N-lobes of Bovine Pathgens
RAMTNLRQCSTSKLL

(19) United States
(12) Patent Application Publication
(10) Pub. No.: US 2004/0258695 A1
(43) Pub. Date: Dec. 23, 2004

(54) Transferrin binding peptides and uses thereof

(76) Inventor: Anthony Bernard Schryvers, Calgary (CA)

Correspondence Address: BURNS DOANE SWECKER & MATHIS L L P, POST OFFICE BOX 1404, ALEXANDRIA, VA, 22313-1404, US

(21) Appl. No.: 10/769,514

(22) Filed: Jan. 30, 2004

Related U.S. Application Data
(60) Provisional application No. 60/444,113, filed on Jan. 31, 2003.

(51) Int. Cl. A61K 39/40; C07K 16/46

(52) U.S. Cl. 424/178.1; 530/391.1

(57) ABSTRACT

The present invention relates to transferrin-binding molecules, particularly peptides, that can (a) bind to regions of transferrin that are recognized by a bacterial transferrin binding protein, and (b) elicit antibodies specifically recognizing the transferrin binding protein. Also provides are compositions, pharmaceutical compositions, and particularly vaccines comprising the molecules, as well as antibodies against the molecules. The molecules can be used to prevent or treat bacterial infections.

This application claims the benefit of U.S. Provisional Patent Application Ser. No. 60/444,113, which was filed Jan. 31, 2003, under 35 U.S.C. 119(e). The entire disclosure of the indicated provisional application is hereby incorporated by reference for all purposes.

### FIELD OF THE INVENTION

This invention relates to molecules, particularly peptides, that are capable of binding transferrin as well as eliciting an immune response against at least one pathogen.

### REFERENCES

U.S. Pat. No. 6,075,181.

U.S. Pat. No. 6,150,584.

Retzer MD, et al. Identification of sequences in human transferrin that bind to the bacterial receptor protein, transferrin-binding protein B. Mol. Microbiol. 32(1):111-121 (1999).

Singh et al. Advances in vaccine adjuvants. Nat. Biotechnol. 17(11):1075-1081 (1999).

Winter G, et al. Man-made antibodies. Nature 349: 293-299 (1991).

All of the publications, patents and patent applications cited herein or in Attachment A are incorporated by reference in their entirety to the same extent as if the disclosure of each individual publication, patent application or patent was specifically and individually indicated to be incorporated by reference in its entirety.

### BACKGROUND OF THE INVENTION

Iron is an essential element for most forms of life due to its role as a cofactor for enzymes and proteins mediating critical functions in energy metabolism and biosynthetic reactions. As a consequence of its importance, its relative insolubility and the potential for free ferric ion to mediate toxic reactions in combination with oxygen, organisms have developed specific systems for its transport, storage and utilization. In the extracellular milieu of the mammalian host, iron is complexed by the glycoprotein transferrin (Tf), which is responsible for transport of iron throughout the body.

Tf effectively lowers the level of free extracellular iron to the extent that growth of microbes is not supported. Thus pathogenic bacteria require mechanisms for acquiring iron from this host glycoprotein in order to survive and cause disease. One mechanism that is present in a number of important Gram negative pathogens of humans and feed animals involves surface receptors that directly bind Tf and mediate removal of iron for its subsequent transport into the cell. Studies with infection models in humans and pigs have demonstrated that these receptors are essential for disease causation. The most extensively studied type of Tf receptor consists of two component proteins, transferrin binding protein A (TbpA) and B (TbpB).

Tf receptors are potentially ideal vaccine targets due to the critical function they mediate and their inherent accessibility at the cell surface. However, a limitation of vaccines based on intact receptor proteins, particularly TbpB, is antigenic variation of these surface antigens. As a result, several proteins are required to provide reasonable coverage against known strains in a given species, and the effectiveness of the vaccine over time is uncertain due to ongoing antigenic variation. Consequently, it is desirable to design a vaccine that is not affected by antigenic variation of the Tf receptors.

### SUMMARY OF THE INVENTION

The basic concept of this invention is that those regions on transferrin receptors from pathogenic bacteria that are involved in binding to transferrin are ideal targets for development of broad-spectrum, long-lasting vaccines. These regions are referred to as “transferrin binding determinants”. Since binding to transferrin is an indispensable function required for survival of the bacteria, transferrin binding determinants are unlikely to be subject to antigenic variation. The present invention thus targets functional epitopes, which are conserved amongst different strains of pathogens and, by virtue of their function, are unlikely to change and evade the immune system.

Furthermore, it has been discovered that the same regions of transferrin bind to the TbpBs from different species of pathogen, indicating that Tf receptors from different species of pathogens share the same, or similar, transferrin binding determinants. Therefore, vaccines comprising transferrin binding determinants can also effectively elicit immune responses to a broad-spectrum of bacteria.

In addition to sequences from the Tf receptors that constitute these transferrin binding determinants, any molecule that is capable of (a) binding to regions of transferrin that are recognized by a bacterial transferrin binding protein, and (b) eliciting antibodies specifically recognizing the transferrin binding protein, will also act as a good vaccine. In particular, mimics of the transferrin binding determinants are also encompassed in the present invention.

Accordingly, one aspect of the present invention provides an isolated molecule capable of:

(a) binding to a region of transferrin that is recognized by a bacterial transferrin binding protein; and

(b) eliciting an antibody to said bacterial transferrin binding protein.

The molecule can be a peptide, an antibody, a recombinant protein, or a conjugate of a peptide and a carrier. Preferably, the molecule binds to a region of the human transferrin that is recognized by a transferrin binding protein of a Gram negative bacterium. The region may comprise a sequence selected from the group consisting of SEQ ID NOs: 1-14 or conservative variants thereof.

In an embodiment of the present invention, the molecule is an isolated peptide comprising a transferrin-binding determinant of a transferrin binding protein of a bacterium, particularly a peptide comprising a sequence selected from the group consisting of SEQ ID NOs: 17, 20, 25, 28, 30, 34, 36, 39, and 48-86 or conservative variants thereof. Alternatively, the peptide may comprise a portion of a sequence selected from the group consisting of SEQ ID NOs: 17, 20, 25, 28, 30, 34, 36, 39, and 48-86 or conservative variants thereof. The portion preferably comprises at least about 9 contiguous amino acids, more preferably at least about 7 contiguous amino acids, still more preferably at least about 5 contiguous amino acids, and most preferably at least about 4 contiguous amino acids, from any one of SEQ ID NOs: 17, 20, 25, 28, 30, 34, 36, 39, and 48-86 or conservative variants thereof.

Another aspect of the present invention provides a vaccine comprising the molecules, particularly the transferrin-binding determinants, as described above. The vaccine is preferably capable of eliciting antibodies that recognize a plurality of different transferrin binding proteins, particularly antibodies that recognize at least two transferrin binding proteins of Gram negative bacteria. More preferably, the vaccine is capable of eliciting antibodies that recognize at least two transferrin binding proteins selected from the group consisting of transferrin binding proteins of Neisseria spp., Haemophilus spp., Moraxella spp., Mannheimia (Pasteurella) spp., Actinobacillus spp., and Staphylococcus spp. Even more preferably, the vaccine is capable of eliciting antibodies that recognize at least two transferrin binding proteins selected from the group consisting of transferrin binding proteins of N. meningitidis, H. influenzae, M. catarrhalis and S. pneumoniae. Most preferably, the vaccine is capable of eliciting antibodies that recognize the transferrin binding proteins of H. influenzae and M. catarrhalis, or the transferrin binding proteins of N. meningitidis and H. influenzae.

Another aspect of the present invention provides an isolated antibody, or a fragment thereof, wherein the antibody or fragment recognizes a plurality of different transferrin binding proteins. The fragment is preferably the Fv, Fab, Fab′, or F(ab′)₂ fragment of the antibody. The antibody may be polyclonal or monoclonal. The antibody or fragment preferably recognizes at least two transferrin binding proteins selected from the group consisting of transferrin binding proteins of Neisseria spp., Haemophilus spp., Moraxella spp., Mannheimia (Pasteurella) spp., Actinobacillus spp., and Staphylococcus spp. More preferably, the antibody or fragment recognizes at least two transferrin binding proteins selected from the group consisting of transferrin binding proteins of N. meningitidis, H. influenzae, M. catarrhalis and S. pneumoniae. Most preferably, the antibody or fragment recognizes the transferrin binding proteins of H. influenzae and M. catarrhalis, or the transferrin binding proteins of N. meningitidis and H. influenzae.

Another aspect of the present invention provides a method of preventing or treating a bacterial infection in a mammal, comprising administering to the mammal an effective amount of a transferrin binding molecule, or an antibody recognizing a transferrin binding protein. The bacterial infection is preferably associated with bacterial meningitis or otitis media. The mammal may be a human, non-human primate, feline, canine, rodent, or domestic animal (such as horse, sheep, cattle and pig). Preferably, the mammal is a human or domestic animal.

Another aspect of the present invention provides a method of identifying a transferrin-binding determinant in a transferrin binding protein, comprising:

(a) providing an overlapping peptide library corresponding to the transferrin binding protein;

(b) determining the activity of each member of the peptide library to bind transferrin; and

(c) identifying overlapping amino acid sequences shared by at least two binding members of the peptide library as transferrin-binding determinants.

The method can be further used for the identification of conserved transferrin-binding determinants, wherein the method further comprises:

(d) determining the activity of the transferrin-binding determinants of (c) in eliciting antibodies that cross-react with a plurality of different transferrin binding proteins; and

(e) identifying the transferrin-binding determinants that can elicit cross-reactive antibodies as conserved transferrin-binding determinants.

### DETAILED DESCRIPTION OF THE INVENTION

The present invention relates to transferrin-binding molecules, particularly peptides, that can (a) bind to regions of transferrin that are recognized by a bacterial transferrin binding protein, and (b) elicit antibodies specifically recognizing the transferrin binding protein. Since transferrin binding proteins are essential for survival of bacterial pathogens, regions that mediate binding of transferrin binding proteins to transferrin (e.g., transferrin binding determinants) are unlikely to change in the event of antigenic variation. Moreover, transferrin binds to different species of bacteria using the same regions, indicating the transferrin binding determinants are conserved amongst different species of bacteria. Accordingly, transferrin-binding molecules, particularly transferrin binding determinants, can act as broad-spectrum, long-lasting vaccines. In addition to the description herein, Attachment A is part of the disclosure and is herein incorporated by reference in its entirety.

Prior to describing the invention in further detail, the terms used in this application are defined as follows unless otherwise indicated.

Definitions

A “transferrin binding determinant” refers to a region in a transferrin binding protein that binds to transferrin. Preferably, the transferrin binding determinant contains about 50 amino acids or less. The length of the transferrin binding determinant is more preferably about 30 amino acids or less, still more preferably about 20 amino acids or less, and most preferably about 10 amino acids or less.

A “conserved transferrin binding determinant” is a transferrin binding determinant capable of eliciting an antibody that recognizes transferrin binding proteins from at least two different species or strains of bacteria.

A “transferrin binding protein” is a protein that binds transferrin. The protein may comprise a single polypeptide or multiple polypeptides. For example, bacterial transferrin receptors are transferrin binding proteins. Many bacterial transferrin receptors have been found to consist of two polypeptides, termed “transferrin binding protein A” and “transferrin binding protein B” (often referred to as ThpA and TbpB, respectively), each of which is also a transferrin binding protein.

A “conservative variation,” as used herein, is the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like.

A “peptide”, as used herein, may contain any number of amino acids. A peptide consists of preferably about 150 amino acids or less, more preferably about 100 amino acids or less, still more preferably about 50 amino acids or less, even more preferably about 30 amino acids or less, and most preferably about 20 amino acids or less. A peptide may contain functional groups of other biological molecules such as carbohydrates, lipids, or nucleic acids. Therefore, peptides include glycopeptides, phosphorylated peptides, acetylated peptides, and the like.

An “antibody” is a protein molecule that reacts with a specific antigen and belongs to one of five distinct classes based on structural properties: IgA, IgD, IgE, IgG and IgM.

An “immune response” is the development in the host of a cellular and/or antibody-mediated immune response to an immunogen. Such a response may consist of the production of one or more of the following: antibodies, B cells, helper T cells, suppressor T cells, and/or cytotoxic T cells directed specifically to the immunogen.

A “vaccine” is a composition comprising at least one molecule (“vaccine antigen” or “vaccine immunogen”) that is capable of eliciting in an animal an immune response that reduces or eliminates pathogenic infections. In particular, a vaccine is capable of eliciting an immune response that prevents pathogenic infections.

“Elicit” or “stimulate” an immune response is to cause an immune response by exposing an immune system to an immunogen.

An “immunogen” is a molecule that is capable of eliciting an immune response in an animal.

“Prevent a pathogenic infection” means to prevent, completely or partially, the development of a pathogenic infection.

“Treat a pathogenic infection” means to reduce, completely or partially, the symptoms of a pathogenic infection after the onset of the pathogenic infection.

An “effective amount” is an amount that is sufficient to achieve the intended purposes. For example, an effective amount of a vaccine to treat an infection is an amount of the vaccine sufficient to elicit an immune response in the recipient of the vaccine to reduce, completely or partially, the symptoms of the infection after the onset of the infection.

An “overlapping peptide library corresponding to a protein” is a collection of peptides, each of which consists of a consecutive stretch of amino acids from the protein, wherein each stretch of amino acids overlaps with the amino acid stretch of at least one other peptide in the library. Collectively, the peptides of the library cover the entire sequence of the protein, or region of the protein, of interest.

“Cross-reactivity” of an antibody is the ability of the antibody to recognize at least two different immunogens. The percentage of cross-reactivity of an antibody for two or more immunogens can be determined by any method established in the art. For example, cross-reactivity can be determined by measuring the ability of one immunogen to inhibit binding of another immunogen to the antibody. Alternatively, cross-reactivity can be determined by measuring the binding affinity of the antibody to each of the immunogens, respectively, and expressing each affinity as a percentage of the highest affinity. It should be noted that binding affinity can be measured as relative binding affinity, and a determination of the binding constant is not necessary. For instance, binding affinity can be measured using an enzyme linked immunosorbent assay (ELISA), and the reading of the bound enzyme activity can be used as the relative binding affinity.

Transferrin (Ti) and Transferrin Binding Proteins (Tbps)

Transferrins (Tfs) are a family of bilobed, monomeric glycoproteins that sequester iron to the extent that levels of free iron are insufficient to support pathogenic growth. The main function of transferrin is the transport of Fe³⁺ iron through the body to cells requiring iron. The iron is transported to the cytosol by receptor-mediated endocytosis.

The two lobes of Tf are almost identical in overall structure. Each lobe in turn is made up of two equally sized domains that are connected by a central hinge region. A cleft located between the domains in each lobe serves as the binding site for the metal ion and a carbonate anion. Each lobe of Tf is able to bind one molecule of Fe³⁺, with a binding constant of about 10²⁰. Due to the high binding affinity and a normal state of 30% iron saturation, it is a very effective iron scavenging protein in the serum. The spare iron binding capacity is crucial to sequester any free iron that may be released from lysed cells or freed as a result of infection.

The bacterial Tf receptor typically consists of two iron-repressible outer membrane proteins, transferrin binding proteins A and B (TbpA and TbpB). TbpA is an integral outer membrane protein that is thought to serve as a channel for the transport of iron across the outer membrane in a TonB-dependent fashion. TbpA is required for the utilization of iron from Tf but either of the binding proteins (A or B) can bind iron-loaded Tf.

TbpA is a homologue of the transmembrane siderophore receptors FhuA and FepA, for which the structures have been determined. The structure consists of 22 anti-parallel β-strands that form a barrel or pore through the outer membrane, the amino-terminus of the protein forms a plug in the center of the barrel. These structures have been used as a basis for development of topology models for TbpA. A pentameric sequence called a TonB box is present in the plug region of FhuA, FepA and TbpA. This sequence is thought to be the point of protein interaction for the periplasmic protein TonB which transduces energy from the inner to the outer membrane, thus allowing molecules like iron to move against their concentration gradient into the cell.

TbpB is a peripheral outer membrane lipoprotein anchored to the membrane by N-terminal linked fatty acids and is largely exposed to the extracellular environment. Alignments of the predicted amino acid sequences of TbpB proteins from different organisms show several regions of homology between the N- and C-terminal halves of the proteins (Retzer et al., 1999). This homology is present in spite of the variable sequence and size between the TbpB proteins, with molecular masses ranging from about 65 kDa to more than 85 kDa. The homology between the protein halves suggests that TbpB might have a bilobed structure analogous to Tf, which is supported by the ability to produce separate recombinant lobes capable of binding Tf. Overlapping peptide libraries of human transferrin (hTf) N-lobe and C-lobe have shown that sequentially homologous peptides in each of the hTf libraries bind both the N- and C-terminal lobes of TbpB from the human pathogens M. catarrhalis and N. meningitidis, confirming that they are functional homologues (Retzer et al., 1999).

The Tf-TbpB interaction involves two lobe-lobe interactions that appear to be similar. Evidences suggest that the TbpB N-lobe may bind to the hTf C-lobe and the TbpB C-lobe would thus be expected to bind to the hTf N-lobe. Recombinant TbpB N-lobe from the human pathogens M. catarrhalis and N. meningitidis preferentially binds to chimeric proteins containing the C-lobe and not the N-lobe of human transferrin (Retzer et al., 1999). Since the C-lobe of Tf is preferentially recognized by TbpA, it likely is proximal to the outer membrane and would be positioned appropriately to interact with the membrane anchored N-lobe of TbpB.

It should be pointed out that transferrin and transferrin binding proteins appear to be species specific. For example, transferrin binding proteins from human bacterial pathogens bind to human transferrin but not bovine transferrin, and vice versa. In fact, transferrin may be an important factor in bacterial host specificity.

Transferrin Binding Molecules

The present invention provides transferrin-binding molecules, particularly peptides, that can (a) bind to regions of transferrin that are recognized by a bacterial transferrin binding protein, and (b) elicit antibodies specifically recognizing the transferrin binding protein. The molecules may be, by way of example, peptides corresponding to transferrin binding determinants (regions of the bacterial transferrin protein that bind transferrin), or mimics of transferrin binding proteins.

Transferrin binding determinants can be identified by any method established in the art. For example, as demonstrated in Example 1, an overlapping peptide library corresponding to TbpB of M. catarrhalis was constructed. Each peptide in this library was used to bind labeled human transferrin in order to identify the peptides that are capable of binding transferrin. The peptides that bind transferrin were compared to one another, and any overlapping sequence shared by at least two binding peptides is particularly useful in the present invention. Thus, a number of peptides were found to bind transferrin, and eight sequences (SEQ ID NOs: 12-14, 17, 20, 25, 28, 30, 34, 36, 39) have been identified as core sequences for binding by transferrin binding protein to transferrin. These regions are unlikely to be changed if antigenic variation occurs, thus providing epitopes that can be used to prepare long-lasting vaccines. Similarly, transferrin binding determinants for Tbps of N. meningitidis and H. influenzae (Example 3) and the bovine pathogens M. haemolytica and H. somnus (Example 4) have also been identified.

It has been shown that transferrin binding proteins of different species of bacteria recognize essentially the same regions in human transferrin (Retzer et al., 1999), suggesting that transferrin binding determinants are conserved among different species. Accordingly, it is contemplated that the transferrin binding determinants identified in one species or strain of bacteria are similar in some sense to transferrin binding determinants of other species or strains of bacteria. The transferrin binding determinants may be similar in amino acid sequence and/or conformation. For example, a transferrin binding determinant from M. catarrhalis (GPVGGVFYNGTT, SEQ ID NO: 20) shares the sequence PVGGV (SEQ ID NO: 87) with a transferrin binding determinant from H. influenzae (SALPVGGVATYKGTW, SEQ ID NO: 54). Different transferrin binding determinants may also share common non-contiguous amino acids, or even common conformations formed by distinct amino acid sequences. These shared sequences and conformations are of particular interest in the present invention.

Since the transferrin binding determinants from different species/strains share common sequences and/or conformation, antibodies raised against transferrin binding determinants from one species/strain will cross-react with the transferrin binding proteins from another species/strain as well. Accordingly, transferrin binding determinants can be used to generate cross-reacting antibodies or be used in broad-spectrum vaccines.

In order to improve cross-reactivity of the resultant antibodies, a sequential immunization strategy can be used. Thus, an animal can be immunized sequentially with transferrin binding determinants from different species or strains of bacteria. Since a booster immune response is only induced for those epitopes that are common in sequential immunization, antibodies with relatively high cross-reactivity are selectively enriched. Sequential immunization can also be practiced with intact transferrin binding proteins or portions thereof (particularly the N-lobe or C-lobe) from different bacterial species/strain to produce cross-reactive antibodies or immune responses.

Cross-reactivity of the antibodies is preferably at least about 10%, more preferably at least about 20%, 30%, 40%, 50%, 60%, 70% or 80%, and most preferably at least about 90% between any two bacterial species or strain. Preferably, the antibody cross-reacts with the transferrin binding proteins from two different Gram negative bacteria. More preferably, the antibody cross-reacts with the transferrin binding proteins from at least two species selected from the group consisting of Neisseria spp., Haemophilus spp., Moraxella spp., Mannheimia (Pasteurella) spp., Actinobacillus spp., and Staphylococcus spp. Still more preferably, the antibody cross-reacts with the transferrin binding proteins from at least two species selected from the group consisting of N. meningitidis, H. influenzae, M. catarrhalis and S. pneumoniae. Most preferably, the antibody cross-reacts with the transferrin binding proteins from at least two strains selected from the group consisting of H. influenzae and M. catarrhalis or the group consisting of N. meningitidis and H. influenzae.

An antibody that exhibits a particularly high cross-reactivity (e.g., at least about 50%) between two different transferrin binding proteins can be used to define an epitope that can be included in a vaccine as a vaccine antigen. The epitope can also be used to generate antibodies against these two transferrin binding proteins. This epitope will not necessarily share the same amino acid sequence with any transferrin binding determinant in either of the two transferrin binding proteins. However, the epitope is expected to have similar conformation or amino acid sequence, contiguous or non-contiguous, with the transferrin binding determinants of both of the two transferrin binding proteins. The epitope can be identified, for example, by screening a random peptide library with the antibody of high cross-reactivity.

The present invention further provides mimics of transferrin binding proteins. A mimic of a transferrin binding protein is a protein that contains regions with the same structural characteristics as at least one transferrin binding determinant of the transferrin binding protein. The mimic binds to transferrin, and is capable of eliciting an immune response against the particular transferrin binding protein.

A preferred mimic is an antibody molecule. The mimic can be prepared, for example, by sequential immunization with transferrin or regions of transferrin that bind transferrin binding proteins (Tbp binding sequences). The Tbp binding sequences in human transferrin are listed in Table 1 below:

TABLE 1
Sequences in human transferrin (hTf)
that bind Tbp

| | | |
|---|---|---|
| SEQ ID NO | Location in hTf | Sequence |

| | | | |
|---|---|---|---|
| SEQ ID NO:1 | N-lobe | 101-108 | VKKDSGFQ |
| SEQ ID NO:2 | N-lobe | 109-115 | MNQLRGK |
| SEQ ID NO:3 | N-lobe | 117-127 | SCHTGLGRSAG |
| SEQ ID NO:4 | N-lobe | 185-195 | YFGYSGAFKCL |
| SEQ ID NO:5 | N-lobe | 245-259 | QVPSHTVVARSMGGK |
| SEQ ID NO:6 | N-lobe | 273-287 | HFGKDKSKEFQLFSS |
| SEQ ID NO:7 | N-lobe | 313-327 | MYLGYEYVTATRNLR |
| SEQ ID NO:8 | C-lobe | 433-447 | KKSASDLTWDNLKGK |
| SEQ ID NO:9 | C-lobe | 461-475 | NIPMGLLYNKINHCR |
| SEQ ID NO:10 | C-lobe | 497-511 | LCMGSGLNLCEPNNK |
| SEQ ID NO:11 | C-lobe | 513-527 | GYYGYTGAFRCLVEK |
| SEQ ID NO:12 | C-lobe | 585-591 | HAVVTRK |
| SEQ ID NO:13 | C-lobe | 613-623 | TDCSGNFCLFR |
| SEQ ID NO:14 | C-lobe | 649-663 | KYLGEEYVKAVGNLR |

To identify mimics of transferrin binding proteins, antibodies raised against Tbp binding sequences are first screened for binding to transferrin or Tbp binding sequences. The antibodies are then tested for their binding activity to peptides of an overlapping peptide library corresponding to transferrin. A mimic of a transferrin binding protein should bind to transferrin with the same pattern as the transferrin binding protein itself. Therefore, antibodies that bind to the same peptides in the transferrin overlapping peptide library as the particular transferrin binding protein of interest are identified as mimics. The mimics can be used to raise antibodies against transferrin binding proteins or be used in vaccines.

Compositions and Pharmaceutical Compositions

Compositions comprising the transferrin-binding molecules of this invention are also provided. The compositions can be used to elicit an immune response in an animal for at least two purposes. Where the composition acts as a vaccine by eliciting an immune response in the animal, the resulting antibodies or T-cell mediated immunity can protect the animal from a subsequent attack involving the same epitopes (active immunity). Alternatively, the composition can be used to produce antibodies which can be used as a research tool, or administered to a second animal to protect the second animal from a subsequent attack involving the same epitopes (passive immunity).

To augment the immune response elicited, it may be preferable to couple the transferrin-binding molecule, especially smaller peptides (e.g., those containing about 50 amino acids or less), to a carrier. The carrier is preferably a protein or polysaccharide, and more preferably a protein. Coupling techniques are well known in the art.

In addition, the transferrin-binding molecules or their conjugates with carriers may be further mixed with adjuvants to elicit an immune response, as adjuvants may increase immunoprotective antibody titers or cell mediated immunity response. Such adjuvants may include, but are not limited to, Freunds complete adjuvant, Freunds incomplete adjuvant, aluminum hydroxide, dimethyldioctadecyl-ammonium bromide, Adjuvax (Alpha-Beta Technology), Inject Alum (Pierce), Monophosphoryl Lipid A (Ribi Immunochem Research), MPL+TDM (Ribi Immunochem Research), Titermax (CytRx), QS21, the CpG sequences (Singh et al., 1999), toxins, toxoids, glycoproteins, lipids, glycolipids, bacterial cell walls, subunits (bacterial or viral), carbohydrate moieties (mono-, di-, tri-, tetra-, oligo- and polysaccharide), various liposome formulations or saponins. Combinations of various adjuvants may be used with the antigen to prepare the immunogen formulation.

The composition may be administered by various delivery methods including intravascularly, intraperitoneally, intramuscularly, intradermally, subcutaneously, orally, nasally or by inhalation. The composition may further comprise a pharmaceutically acceptable exicipient and/or carrier. Such compositions are useful for immunizing any animal which is capable of initiating an immune response, such as primate, rodent, bovine, ovine, caprine, equine, leporine, porcine, canine and avian species. Both domestic and wild animals may be immunized. The exact formulation of the compositions will depend on the particular peptide or peptide-carrier conjugate, the species to be immunized, and the route of administration.

The antibodies produced against a transferrin-binding molecule can be included in a pharmaceutical composition and administered to an animal. The pharmaceutical composition typically comprises a pharmaceutically acceptable carrier, and may include pharmaceutically acceptable excipients. The pharmaceutical composition can be administered intravascularly, intraperitoneally, intramuscularly, intradermally, subcutaneously, orally, nasally or by aerosol inhalation. Preferably the pharmaceutical composition is administered intravascularly, intramuscularly, nasally or by aerosol inhalation.

Also encompassed by the present invention are antibodies, particularly monoclonal antibodies, which are derived from the antibodies produced against a transferrin-binding molecule of this invention. In particular, hybridomas can be generated using a transferrin-binding molecule of this invention, and recombinant derivative antibodies can be made using these hybridomas according to well-known genetic engineering methods (for a review, see Winter et al., 1991). For example, the DNA fragment coding for the variable regions of the monoclonal antibodies can be obtained by polymerase chain reactions (PCR). The PCR primers can be oligonucleotides which are complementary to the constant regions of the heavy chain or light chain, and the PCR template can be the total cDNA or genomic DNA prepared from the hybridomas. Alternatively, a cDNA library can be prepared from the hybridomas and screened with probes which correspond to the constant regions of immunoglobulin heavy chain or light chain to obtain clones of the heavy chain or light chain produced by the particular hybridoma.

Subsequently, the DNA fragment for the variable regions can be inserted into an expression vector and joined in frame with the cDNA sequences of a selected constant region. The constant region can be the human constant sequences to make humanized antibodies, the goat constant sequences to make goat antibodies, the IgE constant sequences to make IgE which recognizes the transferrin-binding molecule, and the like. Thus, antibodies with the same antigen recognition ability but different constant regions can be produced. Of particular interest are humanized antibodies, which can be used as therapeutic agents against a disease associated with the cognate antigen in humans without eliciting an undesired immune response against the humanized constant region.

Other methods known in the art to humanize antibodies or produce human antibodies can be utilized as well, including but not limited to the xenomouse technology developed by Abgenix Inc. (U.S. Pat. Nos. 6,075,181; 6,150,584) and the methods developed by Biovation, Bioinvent Intematioal AB, Protein Design Labs., Applied Molecular Evolution, Inc., ImmGenics Pharmaceuticals Inc., Medarex, Inc., Cambridge Antibody Technology, Elan, Eos Biotechnology, MedImmune, MorphoSys or UroGensys Inc. Likewise, other methods known in the art to screen human antibody secreting cells can also be utilized.

The formulation for the composition, comprising either a transferrin-binding molecule or an antibody against a transferrin-binding molecule, will vary depending on factors such as the administration route, the size and species of the animal to be administered, and the purpose of the administration. Suitable formulations for use in the present invention can be found in Remington's Pharmaceutical Sciences.

The pharmaceutical compositions described herein are useful in the treatment or prevention of bacterial infections, particularly those associated with Gram negative bacteria. For humans, these diseases include bacterial meningitis and otitis media.

Bacterial meningitis is caused by H. influenzae (capsular serotype b, Hib), N. meningitidis (predominant capsular serogroups are A, B, C, Y, W-135) and Streptococcus pneumoniae (many capsular types). First generation capsule-based vaccines for these bacteria were only moderately effective and specifically not useful in young children. Conjugate capsular vaccines (carbohydrate capsule conjugated to a protein carrier) for Hib has proven to be quite effective in childhood immunization programs. Conjugate capsular vaccines for the major capsular groups of N. meningitidis (except for group B) and prevalent capsular types in S. pneumoniae have been developed. Although these multivalent conjugate capsular vaccines are costly and only provide partial protection against the infection, they are being incorporated into childhood immunization programs. The inability to develop a conjugate capsular vaccine against group B N. meningitidis (NmB) is a significant deficiency, since the majority of cases of meningococcal infection in OECD countries are caused by bacteria with this capsule type.

Since the vaccine antigens contemplated herein target conserved functional regions of the transferrin binding protein B across all strains of H. influenzae and N. meningitidis, an effective vaccine against bacterial meningitis can be developed with broader protection and a longer life than current products (e.g., Meningitec (Wyeth), Menjugate (Chiron), NeisVacC (Baxter), Mencevax (GlaxoSmithKline), and Menomune-A/C/Y/W-135 (Aventis-Pasteur)).

Otitis media is caused by non-typable H. influenzae, Moraxella catarrhalis and Streptococcus pneumoniae. Major vaccine companies are directing significant efforts towards development of an otitis media vaccine, which is proving to be complicated because of the apparent need for multiple components for each pathogen and because of antigenic variation. It is anticipated that a single vaccine antigen contemplated herein will effectively immunize a subject against at least two of these three major pathogens. An additional antigen for S. pneumoniae may be required. The vaccine product will require relatively few components and will have an extended product life.

The development of a single vaccine for the prevention of both bacterial meningitis and otitis media is also contemplated. This would comprise antigens based on the conserved binding domains of the transferrin binding protein B for H. influenza (all strains), N. meningitidis (all strains), and M. catarrhalis plus an additional antigen for S. pneumoniae.

In addition, the same strategies and approaches could be used to develop antibodies and vaccines against veterinary pathogens, particularly those for respiratory infections in cattle and pigs. As shown in Example 4, transferrin binding determinants have also been identified for two bovine pathogens and many are common between the two pathogens. Therefore, long-lasting and/or broad-spectrum vaccines can be developed based on transferrin binding molecules as described herein.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

° C.=degree Celsius

hr=hour

min=minute

sec=second

μM=micromolar

mM=millimolar

M=molar

ml=milliliter

μl=microliter

mg=milligram

μg=microgram

DMEM=Dulbecco's modified Eagle's medium

FBS=fetal bovine serum

MEM=modified Eagle's medium

PBS=phosphate buffered saline

HANKS=Hanks balanced salt solution

OECD=Organization of Economic Cooperation and Development

### Material and Methods

Bacterial Strains and Plasmids

Protein expression was carried out in Escherichia coli strain ER2507 (#801-J) or ER2508 (#801-K) from New England BioLabs. These strains contain a deletion in the malE gene and thus do not produce native Mbp. Strain ER2508 (#801-K) also contains a Lon protease mutation, and was used for expressing the truncations encoded by segments 1 (no binding regions) and segments 1-2 (binding region 1 only), due to reduced degradation of the truncated proteins during storage. All cloning and expression steps were carried out with the pMal-c2 plasmid (New England BioLabs) that provides in-frame fusions with the malE gene. E. coli cells containing the plasmids were maintained on Luria-Bertani (LB) medium (Gibco BRL) containing 100 μg/ml⁻¹ ampicillin (Sigma). The expression of Mbp or the Mbp fusion proteins was induced by the addition of 200 μM isopropyl-β-D-thiogalactoside (IPTG) to the medium and incubation for 3 h at 37° C. before harvest.

Construction of Overlapping Peptide Libraries

Generation of the overlapping peptide libraries representing human transferrin or the TbpB N-lobe regions from different bacteria was by the ‘SPOTSCAN’ method (Genosys Biotechnologies) (13). Essentially the library consisted of linear peptides 15 amino acids in length that were immobilized onto a cellulose membrane using 9-flurorenylmethoxycarbonyl (Fmoc) amino acid-based synthesis. One membrane was used to generate a library of the C-lobe of hTf consisting of ninety-six 15 amino acid peptides with an 11 amino acid overlap. The overlapping hTf peptide represented amino acids 285 to 679 of the intact hTf protein (accession # NP_{—}001054). The first peptide library representing TbpB N-lobe from M. catarrhalis 4223 (accession #AAC34277) consisted of thirty-two 15 amino acid peptides with a 4 amino acid overlap and included N-lobe amino acids 12-421. The amino acid numbers refer to the amino acids in the predicted mature, processed protein (i.e. the N-terminal cysteine is amino acid 1). The second peptide library representing M. catarrhalis 4223 TbpB N-lobe peptides included amino acids 27-421 but with an 11 amino acid overlap.

Production of Proteins

TbpB truncations were generated by PCR-based cloning into the pMal expression system (New England BioLabs) as described previously (33). Essentially, amplified tbpB gene segments were subcloned into the pMal-c2 expression vector to generate an in frame fusion with the malE gene. Intact Moraxella catarrhalis 4223 TbpB consisted of amino acids 3-680 where 1 represents the N-terminal cysteine of the mature processed protein. Amino acids 1 and 2 from the mature protein were removed to ease cloning and subsequent expression. The N-terminal half of this gene consisted of amino acids 3 to 421. The truncated TbpB proteins were expressed as maltose binding protein fusions in E. coli ER2507 or ER2508 (for segments 1 and 1-2) and purified by amylose affinity chromatography.

Two different methods for labeling proteins for library screening were used. Iron loaded human Tf (Sigma) was labeled by chemical cross-linking (using 3-maleimidobenzoic acid-N-hydroxysuccinimide ester) to a beta-galactosidase (β-gal) reporter enzyme as described elsewhere (18). This was used as a probe for calorimetric detection on the composite peptide library. The second method involved conjugating the truncations of the N-lobe of TbpB from M. catarrhalis 4223 to horseradish peroxidase (HRP). HRP conjugated proteins can be detected by chemiluminescence, providing a much more sensitive screen. The truncations were conjugated to HRP using the Linx HRP Rapid Protein Conjugation Kit (Invitrogen). These labeled fusion proteins were then used as a probe for chemiluminescent detection.

In addition, HRP conjugated hTf (Jackson ImmunoResearch) was prepared and used to probe the M. catarrhalis TbpB N-lobe peptide library.

Screening Peptide Libraries

Binding experiments of hTf to the first peptide library was by the calorimetric method (Genosys Biotechnologies). Essentially hTf in blocking solution (10% Blocking buffer (Genosys Biotechnologies), 50 mg/ml sucrose, T-TBS, (0.137 M NaCl, 2.68 mM KCl, 50.4 mM Tris-Base, 0.05% Tween-20, pH8.0) was incubated with the membrane containing the peptide library for 4 hours at room temperature. The membrane was then washed sequentially in T-TBS and PBS before detection of bound beta-galactosidase using 5-bromo-4-chloro-3-indoyl-β-D-galactopyranoside (X-gal). Positive binding was determined by the development of a blue spot.

The membrane was then regenerated by the following sequence of washes for 10 minutes each at room temperature on a rocking table: 3 changes of 20 ml of ddH₂O; 3 changes of 20 ml of N, N-Dimethylformamide (DMF) (Sigma); 2 changes of ddH₂O; 3 changes of regeneration buffer A (8.0 M urea, 1% sodium dodecyl sulphate, 0.1% 2-mercaptoethanol); 3 changes of regeneration buffer B (50% ethanol, 10% acetic acid in ddH₂O). Finally the membrane was washed with 2 changes of 20 ml of methanol, allowed to air dry and stored at −20° C. The same membrane could be reused up to 5 times if the regeneration protocol was followed carefully.

For increased sensitivity, the TbpB N-lobe truncations from M. catarrhalis were conjugated to HRP for production and detection of chemiluminescent product. The membrane was rinsed with methanol and washed with 3 changes (5 minutes each) of TBS (0.137 M NaCl, 2.68 mM KCl, 50.4 mM Tris-Base, pH=8.0) on a rocking platform. The membrane was blocked by rocking at room temperature in blocking solution consisting of 20 ml of 0.5% Skim milk powder (BioRad) in TBS for 2.5 hours. The membrane was washed with T-TBS 3 times before being incubated with one of the HRP conjugated truncations diluted 1:1000 in skim milk blocking solution for 4 hours at room temperature. The membrane was then washed again 3 times with T-TBS, drained and incubated with 10 ml of Lumiglo mixture (Kirkegaard and Perry Laboratories) for 5 minutes. It was then exposed to X-omat Blue XB-1 X-Ray film (Kodak) for at least 30 seconds before developing. Positive peptides show up as dark spots on a light background.

### Example 1

### Identification of Transferrin-Binding Determinants of Transferrin Binding Protein B from Moraxella catarrhalis

A prior study demonstrated at least six regions on each hTf lobe bound to TbpBs from N. meningitidis and M. catarrhalis (34). This infers that each lobe of TbpB should possess six complementary binding regions. Thus a strategy for demonstrating these binding determinants was adopted that included (i) identification of hTf-binding peptides by probing peptide libraries from the TbpB N-lobe, and (ii) generating TbpB N-lobe truncations lacking the identified peptides to demonstrate loss of binding to peptides in the hTf library. M. catarrhalis was selected for our experiments since recombinant TbpBs and TbpB subfragments (lobes) from this species were the most stable and retained the strongest binding activity.

Initially, an overlapping peptide library representing the TbpB N-lobe of M. catarrhalis was prepared and probed for binding to labeled hTf. This library was comprised of thirty-two 15 mer peptides with a 4-amino acid overlap representing amino acids 12-421 of the mature M. catarrhalis TbpB. The library was screened for binding to β-galactosidase (β-gal) conjugated hTf (β-gal-hTf ) and a number of positive-binding peptides were detected with varying intensity of colored product. Three strongly positive peptides (#80, 90 and 92), two moderately positive peptides (#65 and 83) and one weakly positive peptide (#85) were identified that represent the six binding peptides (Table 2) for our truncation analysis (see Example 2). As a negative control, the library was screened with β-galactosidase alone to demonstrate that the reactivity of the peptides recognized by labeled hTf was due to binding of hTf.

TABLE 2
Human transferrin binding by peptides from
the M. catarrhalis TbpB N-lobe

| | Peptide | | |
|---|---|---|---|
| Binding Peptide | Library | Amino Acid | |
| Region | (Spot #) | Sequence | Overlap Sequence |
| 1 | 1 (65) | mgygmalskinlhnr | mgygmalskinlhn |
| | | (SEQ ID NO: 15) | (SEQ ID NO:17) |
| | 2 | amgygmalskinlhn | |
| | | (SEQ ID NO: 16) | |
| 2 | 1 (80) | gpvggvfyngtttak | gpvggvfyngtt |
| | | (SEQ ID NO: 18) | (SEQ ID NO: 20) |
| | 2 | wnlgpvggvfyngtt | |
| | | (SEQ ID NO: 19) | |
| 3 | 1 (83) | fmtdvanrrnrfsev | fmtdva |
| | | (SEQ ID NO: 21) | (SEQ ID NO: 25) |
| | 2 | avkykghwdfmtdva | |
| | | (SEQ ID NO: 22) | |
| | | kghwdfmtdvanrrn | |
| | | (SEQ ID NO: 23) | |
| | | dfmtdvanrrnrfse | |
| | | (SEQ ID NO: 24) | |
| 4 | 1 (85) | agwyygasskdeynr | agwyyg |
| | | (SEQ ID NO: 26) | (SEQ ID NO: 28) |
| | 2 | fsevkensqagwyyg | |
| | | (SEQ ID NO: 27) | |
| 5 | 1 (90) | fsnlqdrhkgnvtkt | fsnl |
| | | (SEQ ID NO: 29) | (SEQ ID NO: 30) |
| | 2 | nfkekkltgklfsnl | gkfsnl |
| | | (SEQ ID NO: 31) | (SEQ ID NO: 34) |
| | | kkltgklfsnlqdrh | |
| | | (SEQ ID NO: 32) | |
| | | gklfsnlqdrhkgnv | |
| | | (SEQ ID NO: 33) | |
| 6 | 1 (92) | danihgnrfrgsata | danihgnr |
| | | (SEQ ID NO: 35) | (SEQ ID NO: 36) |
| | 2 | kterydidanihgnr | ydidanihgnr |
| | | (SEQ ID NO: 37) | (SEQ ID NO: 39) |
| | | ydidanihgnrfrgs | |
| | | (SEQ ID NO: 38) | |

To confirm and refine the identification of the binding regions, a second overlapping TbpB N-lobe peptide library was prepared and probed with an HRP-conjugate of hTf. The use of an HRP-conjugate allowed the more sensitive detection of a chemiluminescent product. The second library consisted of ninety-six 15 amino acid peptides with an 11 amino acid overlap, representing amino acids 23 to 421 of the mature M. catarrhalis TbpB. The greater degree of overlap and the use of the more sensitive chemiluminescence detection method increased the chance of detecting binding peptides. Thus a total of 11 binding peptides were detected in this peptide library compared to the 6 binding peptides that were identified in the first library (Table 2). None of these peptides were detected when the library was screened with HRP alone. This second library revealed the same six binding regions as the first library but included overlapping peptides for several regions.

The availability of overlapping positive peptides allowed further definition of the binding determinants, since the common amino acid sequence could be implicated as the recognition sequence (Table 2). Thus, binding region 6, represented by two overlapping peptides in the second library, shared an 11 amino acid hendecapeptide (ydidanihgnr) and, by including the peptide recognized in the first library, an octapeptide (danihgnr) could be implicated as the binding region. Similarly, binding region 3, which is represented by three adjacent overlapping peptides in the second library, tentatively identified a heptapeptide (dfmtdva) sequence which overlaps a hexapeptide sequence (fintdva) identified in the first peptide library.

Three of the binding regions (binding regions 1, 2 and 4, Table 2) were represented by a single peptide from both libraries. Since adjacent peptides in the 2^{nd} library contain 11/15 amino acids in common, it is unlikely that the lack of binding to the neighbouring peptides is solely due to the absence of the amino acids directly involved in the binding interaction, but suggests that conformational or structural features of the peptides were also important for detectable binding. For two of the regions (binding region 1 and 2, Table 2), binding peptides from the composite library effectively provided adjacent peptides with a greater degree of overlap (14/15 for segment 1, 12/15 for segment 2) and thus provided a minor refinement in localizing the binding determinants. The binding peptide from the first peptide library for binding region 4 provided less overlap (6/15), yet retained binding activity for unknown reasons.

### Example 2

### Verification of Binding Determinants using Truncations in the N-Lobe of TbpB

The binding peptides listed in Table 2 were identified by binding to labeled hTf, and, by inference, would be expected to interact with one of the TbpB-binding peptides identified in the hTf peptide library (35). To provide direct evidence for this interaction, one strategy was to eliminate the putative binding determinant from TbpB and determine whether it resulted in a loss of reactivity with one of the TbpB binding peptides from hTf. The approach was to generate a series of truncations of the TbpB N-lobe that sequentially eliminated the segments of TbpB containing hTf-binding determinants and use the truncated proteins to probe the hTf peptide library. Although producing site-directed mutants of the TbpB N-lobe was another alternative, it would have required preparation of a large number of mutants due to the length of the identified peptide regions.

To plan the truncation experiments, the N-lobe of M. catarrhalis was divided into seven segments with the identified binding peptides at their junctions. Segment 1 includes the amino acid sequence up to but not including binding region 1, segment 2 includes binding region 1 and the amino acid sequence up to but not including binding region 2. Continuing with this convention, segment 7 includes binding region 6 and the remaining amino acid sequence to the end of the N-lobe of TbpB.

To generate the TbpB N-lobe truncations, PCR mutagenesis was used to introduce a stop codon and an appropriate restriction site at the junction of the segments. Using a plasmid expressing the functional TbpB N-lobe as a template, PCR amplifications were performed. The PCR products were subcloned into the pMal-c2 vector and the sequence was confirmed. After expression, the resulting maltose binding protein (Mbp) fusion proteins were isolated by amylose affinity chromatography. Initial experiments showed that the smaller expressed truncations that contain binding regions 1 and 2 were degraded more quickly than the other truncations or the intact proteins during storage. In order to make more stable preparations for these truncations, a protease deficient expression strain was used.

Relatively pure and stable preparations of all of the truncations were isolated by amylose affinity chromatography. Each protein was electroblotted onto nitrocellulose and probed with anti M. catarrhalis ThpA/B antibodies. All the bands observed in the Coomassie blue gel reacted with the antibody (data not shown) indicating that they were breakdown products of the fusion protein and not impurities. Solid-phase binding assays using HRP-conjugated hTf to probe immobilized fusion proteins showed an increasing reduction or loss of detectable binding activity with successive truncations. The fusion proteins containing segments 1-4 or less did not yield detectable binding activity in the solid-phase binding assay using the calorimetric substrate, chloronapthol (data not shown). In addition, if the truncations were probed with labeled hTf after SDS-PAGE and electroblotting, even the larger truncated proteins were not detected, similar to what was observed for the TbpB from N. meningitidis (40).

The lack of binding to hTf by some of the TbpB truncations in the screening solid-phase binding assay could have been attributed to many factors including the sensitivity of the calorimetric detection or constraints imposed by immobilizing the protein on the cellulose. Thus they were included for probing the overlapping peptide library of hTf since the format of this assay might permit detection of binding activity by these truncations.

To provide a potentially more sensitive means of detection that would not compromise the lifespan of the peptide libraries, HRP conjugates of the fusion proteins and a chemiluminescent system for detecting binding activity were used. A 15 residue overlapping peptide library representing the C-terminal half of hTf with an 11 amino acid overlap was prepared. This is essentially identical to the library used in a prior study (35) and the intact N-lobe of M. catarrhalis TbpB recognized an almost identical set of peptides (Segments 1-7, Table 3).

The truncation lacking segment 7 bound all of the peptides except the one localized to the C-terminal tail region of hTf (#20, Cyan), suggesting that segment 7 was responsible for binding to the C-terminal tail region (Segments 1-6, Table 3). The truncation lacking both segments 6 and 7 (Segments 1-5, Table 3), lost reactivity to an additional peptide localized to domain 2 (#16, yellow). Although this peptide is quite distant from peptide 20 in the linear amino acid sequence, it is immediately adjacent on the surface of hTf ((35)). Since the binding peptides from segments 6 and 7 are fairly close in the linear amino acid sequence of TbpB, the implication that they bind adjacent regions on the hTf surface seems reasonable.

The results with the subsequent three truncations followed a similar pattern as observed with the first two truncations, removal of an additional segment of TbpB resulted in loss of binding to a peptide that maps on an adjacent position on the hTf surface. Thus the truncation containing segments 1-4 lost reactivity to peptide 13, (Blue), the truncation containing segments 1-3 lost reactivity to peptide 12 (green) and the truncation containing segments 1-2 lost reactivity to peptides 14 and 15 (red). The binding peptides from these segments of TbpB are fairly close in the linear amino acid sequence and map to adjacent regions on the surface of domain 2 of the hTf C-lobe.

The final truncation (segment 1, Table 3) removed a segment containing over 140 amino acids between the binding peptides identified in the TbpB library. This truncation resulted in the loss of binding to two peptides that map to the surface of domain 1 (peptides 18 and 19, brown) which is relatively close to the prior peptides (14 and 15, red) on the surface of domain 2 in the iron-loaded form of hTf. However, these two regions would be substantially further apart in the apo form of the protein in which there is considerable separation of the two domains.

TABLE 3
Identification of TbpB-binding peptides of hTf C-lobe with labeled
TbpB N-lobe truncations

| | | | Binding By Truncations | | | | | | |
|---|---|---|---|---|---|---|---|---|---|
| Binding Peptides | | | Segments Included | | | | | | |
| | | Domain | 1-7 | | | | | | |
| # | Sequence | (Color) | (Intact) | 1-6 | 1-5 | 1-4 | 1-3 | 1-2 | 1 |
| 40 | kksasdltwdnlkgk | 2 | + | + | + | + | − | − | − |
| | | (Green) | | | | | | | |
| 41 | nipmgllynkinhcr | 2 | + | + | + | − | − | − | − |
| | | (Blue) | | | | | | | |
| 42 | lcmgsglnlcepnnk | 2 | + | + | + | + | + | − | − |
| | | (Red) | | | | | | | |
| 43 | gyygytgafrclvek | 2 | + | + | + | + | + | − | − |
| | | (Red) | | | | | | | |
| 44 | chlarapnhavvtrk | 2 | + | + | − | − | − | − | − |
| | | (Yellow) | | | | | | | |
| 45 | gsnvtdcsgnfclfr | 1 | + | + | + | + | + | + | − |
| | | (Brown) | | | | | | | |
| 46 | tdcsgnfclfrsetk | 1 | + | + | + | + | + | + | − |
| | | (Brown) | | | | | | | |
| 47 | kylgeeyvkavgnlr | Tail | + | − | − | − | − | − | − |
| | | (Cyan) | | | | | | | |

### Example 3

### Identification of Transferrin-Binding Determinants of Transferrin Binding Protein B from N. meningitidis and H. influenzae

Overlapping peptide libraries were constructed for the TbpB proteins of N. meningitidis and H. influenzae, and peptides that bind to human transferrin were identified as described in Example 1. The resulting binding peptides are listed below.

Binding peptides for N. meningitidis:

| | |
|---|---|
| FYKHAASEKDFSNKK | SEQ ID NO:48 |
| PSRQLPASGKVIYKG | SEQ ID NO:49 |
| VIYKGVWHFVTDTKK | SEQ ID NO:50 |

Binding peptides for H. influenzae:

| | |
|---|---|
| AALNLFDRNKPSLLN | SEQ ID NO:51 |
| APNSNENRHGQKYVY | SEQ ID NO:52 |
| IQSWSLRDLPNKKFY | SEQ ID NO:53 |
| SALPVGGVATYKGTW | SEQ ID NO:54 |
| YKGTWSFITAAENGK | SEQ ID NO:55 |
| RNSGGGQAYSRRSAT | SEQ ID NO:56 |
| FTVNFGTKKLTGGLY | SEQ ID NO:57 |
| TDANKSQNRTHKLYD | SEQ ID NO:58 |
| GKFLAHDKKVLGVFS | SEQ ID NO:59 |

### Example 4

### Identification of Transferrin-Binding Determinants of Transferrin Binding Protein B from M. haemolytica and H. somnus

Since transferrin and transferrin binding proteins are important for pathogenic bacteria of other mammalian species as well, the present invention can also be used to develop vaccines and antibodies for veterinary uses. These mammalian species include, for example, domestic animals (such as cattle, sheep, horses, and pigs), cats, dogs, rodent, and non-human primates.

Transferrin binding determinants from two bovine pathogens, M. haemolytica and H. somnus, as well as potential conserved transferrin binding determinants derived therefrom, were identified as described in Example 1 and shown in Table 4 below. Briefly, purified TbpB N-lobes from M. haemolytica and H. somnus were labelled with HRP and used to probe the bTf C-lobe overlapping peptide library. The peptides from the library that bound to the indicated TbpB N-lobe are listed in the column below the species name. The common overlap sequence from adjacent peptides probed with M. haemolytica (*), H. somnus (**) or both ( ) TbpB N-lobes are indicated for the indicated regions. None indicates that the peptide did not bind to the TbpB N-lobe from that species.

TABLE 4
bTf Binding Regions of the TbpB N-lobes of Bovine Pathogens

| Peptide | | | Common |
|---|---|---|---|
| # | M. haemolytica | H. somnus | Region |
| 1 | MVKWCAIGHQERTKC | MVKWCAIGHQERTKC | HQERTKC |
| | (SEQ ID NO: 60) | (SEQ ID NO: 60) | (SEQ ID NO: 63) |
| 2 | CAIGHQERTKCDRWS | CAIGHQERTKCDRWS | |
| | (SEQ ID NO: 61) | (SEQ ID NO: 61) | |
| 3 | HQERTKCDRWSGFSG | HQERTKCDRWSGFSG | |
| | (SEQ ID NO: 62) | (SEQ ID NO: 62) | |
| 4 | KTSDANINWNNLKDK | KTSDANINWNN64DK | ANINWNNLKDK |
| | (SEQ ID NO: 64) | (SEQ ID NO: 64) | (SEQ ID NO: 66) |
| 5 | ANINWNNLKDKKSCH | ANINWNNLKDKKSCH | |
| | (SEQ ID NO: 65) | (SEQ ID NO: 65) | |
| 6 | None | NSNERYYGYTGAFRC | RYYGYTGAFRC |
| | | (SEQ ID NO: 67) | (SEQ ID NO: 69) |
| 7 | None | RYYGYTGAFRCLVEK | |
| | | (SEQ ID NO: 68) | |
| 8 | None | NTDGNNNEAWALK | NLKKENF* |
| | | (SEQ ID NO: 70) | (SEQ ID NO: 72) |
| 9 | NNNEAWAKNLKKENF | NNNEAWAKNLKKENF | NNNEAWAKNLK** |
| | (SEQ ID NO: 71) | (SEQ ID NO: 71) | (SEQ ID NO: 74) |
| 10 | AWAKNLKKENFEVLC | None | NLK |
| | (SEQ ID NO: 73) | | (SEQ ID NO: 76) |
| 11 | NLKKENFEVLCKDGT | None | |
| | (SEQ ID NO: 75) | | |
| 12 | CHLARGPNHAVVSRK | CHLARGPNHAVVSRK | HAVVSRK* |
| | (SEQ ID NO: 77) | (SEQ ID NO: 77) | (SEQ ID NO: 80) |
| 13 | RGPNHAVVSRKDKAT | RGPNHAVVSRKDKAT | SRK** |
| | (SEQ ID NO: 78) | (SEQ ID NO: 78) | (SEQ ID NO: 82) |
| 14 | HAVVSRKDKATCVEK | HAVVSRKDKATCVEK | |
| | (SEQ ID NO: 79) | (SEQ ID NO: 79) | |
| 15 | None | SRKDKATCVEKILNK | |
| | | (SEQ ID NO: 81) | |
| 16 | RDDTKCLASIAKKTY | RDDTKCLASIAKKTY | KCLASIAKKTY |
| | (SEQ ID NO: 83) | (SEQ ID NO: 83) | (SEQ ID NO: 85) |
| 17 | KCLASIAKKTYDSYL | KCLASIAKKTYDSYL | |
| | (SEQ ID NO: 84) | (SEQ ID NO: 84) | |
| 18 | RAMTNLRQCSTSKLL | RAMTNLRQCSTSKLL | RAMTNLRQCSTSKLL |
| | (SEQ ID NO: 86) | (SEQ ID NO: 86) | (SEQ ID NO: 86) |

I claim:

1. An isolated molecule capable of:
(a) binding to a region of transferrin that is recognized by a bacterial transferrin binding protein; and
(b) eliciting an antibody to said bacterial transferrin binding protein.

2. The molecule of claim 1 wherein the molecule is an antibody.

3. The molecule of claim 1 wherein the molecule is a recombinant protein or peptide.

4. The molecule of claim 1 wherein the transferrin is human transferrin, and the transferrin binding protein is a transferrin binding protein B from a human Gram negative bacterial pathogen.

5. The molecule of claim 1 wherein the region of transferrin comprises a sequence selected from the group consisting of SEQ ID NOs:1-14.

6. An isolated peptide comprising a transferrin-binding determinant of a transferrin binding protein of a bacterium.

7. The peptide of claim 6 comprising the sequence selected from the group consisting of SEQ ID NOs: 17, 20, 25, 28, 30, 34, 36, 39, and 48-86.

8. A vaccine comprising the molecule of any of claims 1-5, or the peptide of claim 6 or claim 7.

9. The vaccine of claim 8 capable of eliciting antibodies that recognize a plurality of different transferrin binding proteins.

10. The vaccine of claim 8 capable of eliciting antibodies that recognize at least two transferrin binding proteins of Gram negative bacteria.

11. The vaccine of claim 8 capable of eliciting antibodies that recognize at least two transferrin binding proteins selected from the group consisting of transferrin binding proteins of Neisseria spp., Haemophilus spp., Moraxella spp., Mannheimia (Pasteurella) spp., Actinobacillus spp., and Staphylococcus spp.

12. The vaccine of claim 8 capable of eliciting antibodies that recognize at least two transferrin binding proteins selected from the group consisting of transferrin binding proteins of N. meningitidis, H. influenzae, M. catarrhalis and S. pneumoniae.

13. The vaccine of claim 8 capable of eliciting antibodies that recognize the transferrin binding proteins of H. influenzae and M. catarrhalis.

14. The vaccine of claim 8 capable of eliciting antibodies that recognize the transferrin binding proteins of N. meningitidis and H. influenzae.

15. An isolated antibody, or a fragment thereof, wherein the antibody recognizes a plurality of different transferrin binding proteins.

16. The antibody or fragment of antibody of claim 15, wherein the antibody is monoclonal.

17. The antibody or fragment of antibody of claim 15, wherein the antibody is polyclonal.

18. The antibody or fragment of antibody of claim 15, wherein the antibody recognizes at least two transferrin binding proteins selected from the group consisting of transferrin binding proteins of Neisseria spp., Haemophilus spp., Moraxella spp., Mannheimia (Pasteurella) spp., Actinobacillus spp., and Staphylococcus spp.

19. The antibody or fragment of antibody of claim 15, wherein the antibody recognizes at least two transferrin binding proteins selected from the group consisting of transferrin binding proteins of N. meningitidis, H. influenzae, M. catarrhalis and S. pneumoniae.

20. The antibody or fragment of antibody of claim 15, wherein the antibody recognizes the transferrin binding proteins of H. influenzae, M. catarrhalis and S. pneumoniae.

21. The antibody or fragment of antibody of claim 15, wherein the antibody recognizes the transferrin binding proteins of N. meningitidis, H. influenzae, and S. pneumoniae.

22. A method of identifying a transferrin-binding determinant in a transferrin binding protein, comprising:
(a) providing an overlapping peptide library corresponding to the transferrin binding protein;
(b) determining the activity of each member of the peptide library to bind transferrin; and
(c) identifying overlapping amino acid sequences shared by at least two binding members of the peptide library as transferrin-binding determinants.

23. The method of claim 22 useful for the identification of conserved transferrin-binding determinants, wherein the method further comprises:
(d) determining the activity of the transferrin-binding determinants of (c) in eliciting antibodies that cross-react with a plurality of different transferrin binding proteins; and
(e) identifying the transferrin-binding determinants that can elicit cross-reactive antibodies as conserved transferrin-binding determinants.

24. A method for preventing or treating a bacterial infection in a mammal, comprising administering to the mammal an effective amount of the molecule of any of claims 1-5, or the peptide of claim 6 or claim 7.

25. A method for preventing or treating a bacterial infection in a mammal, comprising administering to the mammal an effective amount of an antibody that specifically recognizes the molecule of any of claims 1-5, or the peptide of claim 6 or claim 7.

26. The method of claim 24 or 25 wherein the bacterial infection is associated with a bacterium selected from the group consisting of Neisseria spp., Haemophilus spp., Moraxella spp., Mannheimia (Pasteurella) spp., Actinobacillus spp., and Staphylococcus spp.

27. The method of claim 24 or 25 wherein the bacterial infection is associated with bacterial meningitis or otitis media.
